(19) United States
(12) Patent Application Publication
(10) Pub. No.: US 2012/0022655 A1
(43) Pub. Date: Jan. 26, 2012

(54) ABSORBER DESIGN FOR IMPLANTABLE DEVICE

(75) Inventor: Anton G. Clifford, Mountain View, CA (US)

(73) Assignee: MOXIMED, INC., Hayward, CA (US)

(21) Appl. No.: 12/843,381

(22) Filed: Jul. 26, 2010

(51) Int. Cl. A61F 2/30; B23P 11/00

(52) U.S. Cl. 623/18.11; 29/428

(57) ABSTRACT

A system and method for sharing and absorbing energy between body parts. In one embodiment, the system includes an implantable energy absorbing device having a first part with a piston and arbor arranged parallel to one another and a second part also having a piston and arbor. The piston of the first part is slidable within the arbor of the second part and the piston of the second part is slidable within the arbor of the first part. Further, two springs can be received over the arbors of the first and second part and configured to bias the first and second parts apart. The energy absorbing device is configured to span across a joint.

### BACKGROUND

Various embodiments disclosed herein are directed to structure for body anatomy, and more particularly, towards approaches to devices for unloading joints.

Joint replacement is one of the most common and successful operations in modern orthopaedic surgery. It consists of replacing painful, arthritic, worn or diseased parts of a joint with artificial surfaces shaped in such a way as to allow joint movement. Osteoarthritis is a common diagnosis leading to joint replacement. Such joint replacement procedures are a last resort treatment as they are highly invasive and require substantial periods of recovery. Total joint replacement, also known as total joint arthroplasty, is a procedure in which all articular surfaces at a joint are replaced. This contrasts with hemiarthroplasty (half arthroplasty) in which only one bone's articular surface at a joint is replaced and unincompartmental arthroplasty in which the articular surfaces of only one of multiple compartments at a joint (such as the surfaces of the thigh and shin bones on just the inner side or just the outer side at the knee) are replaced.

Arthroplasty, as a general term, is an orthopaedic procedure which surgically alters the natural joint in some way. Arthroplasty includes procedures in which the arthritic or dysfunctional joint surface is replaced with something else as well as procedures which are undertaken to reshape or realigning the joint by osteotomy or some other procedure. A previously popular form of arthroplasty was interpositional arthroplasty in which the joint was surgically altered by insertion of some other tissue like skin, muscle or tendon within the articular space to keep inflammatory surfaces apart. Another less popular arthroplasty is excisional arthroplasty in which articular surfaces are removed leaving scar tissue to fill in the gap. Among other types of arthroplasty are resection(al) arthroplasty, resurfacing arthroplasty, mold arthroplasty, cup arthroplasty, silicone replacement arthroplasty, and osteotomy to affect joint alignment or restore or modify joint congruity.

The most common arthroplasty procedures including joint replacement, osteotomy procedures and other procedures in which the joint surfaces are modified are highly invasive procedures and are characterized by relatively long recovery times. When it is successful, arthroplasty results in new joint surfaces which serve the same function in the joint as did the surfaces that were removed. Any chodrocytes (cells that control the creation and maintenance of articular joint surfaces), however, are either removed as part of the arthroplasty, or left to contend with the resulting new joint anatomy and injury. Because of this, none of these currently available therapies are chondro-protective.

A widely-applied type of osteotomy is one in which bones beside the joint are surgically cut and realigned to improve alignment in the joint. A misalignment due to injury or disease in a joint related to the direction of load can result in an imbalance of forces and pain in the affected joint. The goal of osteotomy is to surgically re-align the bones at a joint such as by cutting and reattaching part of one of the bones to change the joint alignment. This realignment relieves pain by equalizing forces across the joint. This can also increase the lifespan of the joint. The surgical realignment of the knee joint by high tibial osteotomy (HTO) (the surgical re-alignment of the upper end of the shin bone (tibia) to address knee malalignment) is an osteotomy procedure done to address osteoarthritis in the knee. When successful, HTO results in a decrease in pain and improved function. However, HTO does not address ligamentous instability—only mechanical alignment. Good early results associated with HTO often deteriorate over time.

Other approaches to treating osteoarthritis involve an analysis of loads which exist at a joint and attempts to correct (generally reduce) these loads. Both cartilage and bone are living tissues that respond and adapt to the loads they experience. Within a nominal range of loading, bone and cartilage remain healthy and viable. If the load falls below the nominal range for extended periods of time, bone and cartilage can become softer and weaker (atrophy). If the load rises above the nominal level for extended periods of time, bone can become stiffer and stronger (hypertrophy). Osteoarthritis or breakdown of cartilage due to wear and tear can also result from overloading. When cartilage breaks down, the bones rub together and cause further damage and pain. Finally, if the load rises too high, then abrupt failure of bone, cartilage and other tissues can result.

The treatment of osteoarthritis and other bone and cartilage conditions is severely hampered when a surgeon is not able to control and prescribe the levels of joint load. Furthermore, bone healing research has shown that some mechanical stimulation can enhance the healing response and it is likely that the optimum regime for a cartilage/bone graft or construct will involve different levels of load over time, e.g. during a particular treatment schedule. Thus, there is a need for devices which facilitate the control of load on a joint undergoing treatment or therapy, to thereby enable use of the joint within a healthy loading zone.

Certain other approaches to treating osteoarthritis contemplate external devices such as braces or fixators which attempt to control the motion of the bones at a joint or apply cross-loads at a joint to shift load from one side of the joint to the other. A number of these approaches have had some success in alleviating pain. However, lack of patient compliance and the inability of the devices to facilitate and support the natural motion and function of the diseased joint have been problems with these external braces.

Prior approaches to treating osteoarthritis have also failed to account for all of the basic functions of the various structures of a joint in combination with its unique movement. In addition to addressing the loads and motions at a joint, an ultimately successful approach must also acknowledge the dampening and energy absorption functions of the anatomy. Prior devices designed to reduce the load transferred by the natural joint typically incorporate relatively rigid constructs that are incompressible. Mechanical energy (E) is the action of a force (F) through a distance (s) (i.e., E=F×s). Device constructs which are relatively rigid do not allow substantial energy storage as they do not allow substantial deformations—do not act through substantial distances. For these relatively rigid constructs, energy is transferred rather than stored or absorbed relative to a joint. By contrast, the natural joint is a construct comprised of elements of different compliance characteristics such as bone, cartilage, synovial fluid, muscles, tendons, ligaments, and other tissues. These dynamic elements include relatively compliant ones (ligaments, tendons, fluid, cartilage) which allow for substantial energy absorption and storage, and relatively stiffer ones (bone) that allow for efficient energy transfer. The cartilage in a joint compresses under applied force and the resultant force displacement product represents the energy absorbed by cartilage. The fluid content of cartilage also acts to stiffen its response to load applied quickly and dampen its response to loads applied slowly. In this way, cartilage acts to absorb and store, as well as to dissipate energy.

With the foregoing applications in mind, it has been found to be necessary to develop effective structures for achieving desired load reduction, energy absorption, energy storage, and energy transfer across bones defining a joint.

Therefore, what is needed to treat joint pain is an implant device which addresses both joint movement and varying loads as well as dampening forces and energy absorption provided by an articulating joint while providing a device which includes parts extending across a joint.

The present application satisfies these and other needs.

### SUMMARY

Briefly and in general terms, the present disclosure is directed towards treating diseased or mal-aligned body components. The present disclosure is directed towards methods and devices for treating and preserving body joints.

In one embodiment of treating and preserving body joints, unloading devices are implanted under the patient's skin for relieving joint pain that do not require modification of articular cartilage. In a preferred embodiment, the device is implanted under the patient's skin but outside of the joint capsule. The joint pain may be caused by osteoarthritis.

In one embodiment, the present disclosure addresses the pain associated with joint disease and mal-alignment. It is contemplated that an implantable system for manipulating energy transferred by members defining a joint includes a first attachment structure configured to be attached to a first member of the joint and a second attachment structure configured to be attached to a second member of the joint. The implantable system also includes an energy absorbing device attachable to the first attachment structure and second attachment structure. The energy absorbing device includes a first part having a piston and arbor arranged parallel to one another and a second part having a piston and arbor of substantially the same configuration as the piston and arbor of the first part. When the first and second parts are mated together, the piston of the first part is slidable within the arbor of the second part and the piston of the second part is slidable within the arbor of the first part. The first part and the second part are substantially identical.

Further, the first part and the second part of the implantable system each include a portion of a universal joint and the first and second attachment structures each include a mating portion of the universal joint configured to mate with the first and second parts. The universal joint portion on the first and second parts includes a ball of a ball and socket joint. First and second springs may also be configured to be received over the arbors of the first and second part.

In one embodiment, the implantable system is configured to be entirely implanted within a patient. Also, the implantable system may be configured to be implanted outside of the articular surfaces of the joint. The joint may be a knee joint and the first and second attachment structures are configured to be attached to bones on opposite sides of the knee joint and the energy absorbing device is configured to span across the knee joint.

Another embodiment of an implantable energy absorbing device includes a first part having a piston and arbor arranged parallel to one another and a second part having a piston and arbor of substantially the same configuration as the piston and arbor of the first part. The piston of the first part is slidable within the arbor of the second part and the piston of the second part is slidable within the arbor of the first part. Further, two springs can be received over the arbors of the first and second part and configured to bias the first and second parts apart. The energy absorbing device is configured to span across a joint, and the joint may be a knee joint. In this embodiment, the first and second parts are each unitary parts and are translatable with respect to one another. The first and second parts are substantially identical and rotated 180 degrees with each other to mate in a sliding arrangement.

In one embodiment, the arbors of the first and second parts are formed at least in part as springs. In yet another embodiment, the energy absorbing device includes two springs received over the arbors of the first and second part and configured to bias the first and second parts apart.

Other features and advantages of the present application will become apparent from the following detailed description, taken in conjunction with the accompanying drawings, which illustrate, by way of example, the principles of the invention.

### BRIEF DESCRIPTION OF THE DRAWINGS

FIG. 1A is a perspective view, depicting an embodiment of an energy absorbing system mounted on a knee joint at full extension;

FIG. 1B is a perspective view, depicting the embodiment shown in FIG. 1A with the knee joint flexed to 90°;

FIG. 2 is a perspective view, depicting an absorber part having a piston and an arbor disposed generally parallel to one another; and

FIG. 3 is an exploded view of an energy absorbing system.

### DETAILED DESCRIPTION OF THE PREFERRED EMBODIMENTS

Referring now to the drawings, which are provided by way of example and not limitation, the present disclosure is directed towards apparatus and methods for treating body tissues. In applications relating to the treatment of body joints, the present disclosure seeks to alleviate pain associated with the function of diseased or malaligned members forming a body joint. Whereas the present disclosure is particularly suited to address issues associated with joint degeneration or osteoarthritis, the energy manipulation accomplished by the present disclosure lends itself well to broader applications. Moreover, the present disclosure is particularly suited to treating synovial joints such as the knee, hip, ankle and shoulder. In one aspect of treating and preserving body joints, unloading devices are implanted under the patient's skin for relieving joint pain that do not require modification of articular cartilage. In a preferred aspect, the device is implanted under the patient's skin but outside of the joint capsule.

In one particular aspect, the present embodiments seek to permit and complement the unique articulating motion of the members defining a body joint of a patient while simultaneously manipulating energy being experienced by both cartilage and osseous tissue (cancellous and cortical bone). It has been postulated that to minimize pain in a knee joint, off-loading or absorption of 1-40% of forces, in varying degrees, may be necessary. Variable off-loading or absorption in the range of 5-20% can be a target for certain applications. In certain specific applications, distraction is employed to reduce the loading on articular cartilage and reduce pain.

Some examples of embodiments of implantable systems for manipulating energy transferred by members defining a joint are described in U.S. Patent Publication Nos. 2008/0275555; 2008/0275561; 2008/0275552; and 2009/0014016 each of which is incorporated herein by reference in it's entirety. These implantable systems as well as the system described herein absorb a degree of the forces between body members by the energy manipulating assembly resulting in less force placed on natural body anatomy. In one example, the implantable system for manipulation of energy provides unloading of approximately 20-50 pounds normally experienced by the knee joint.

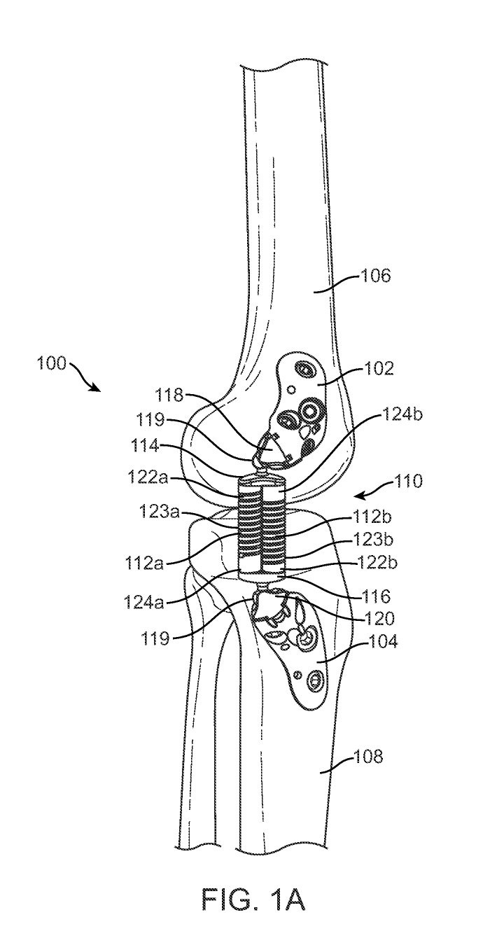

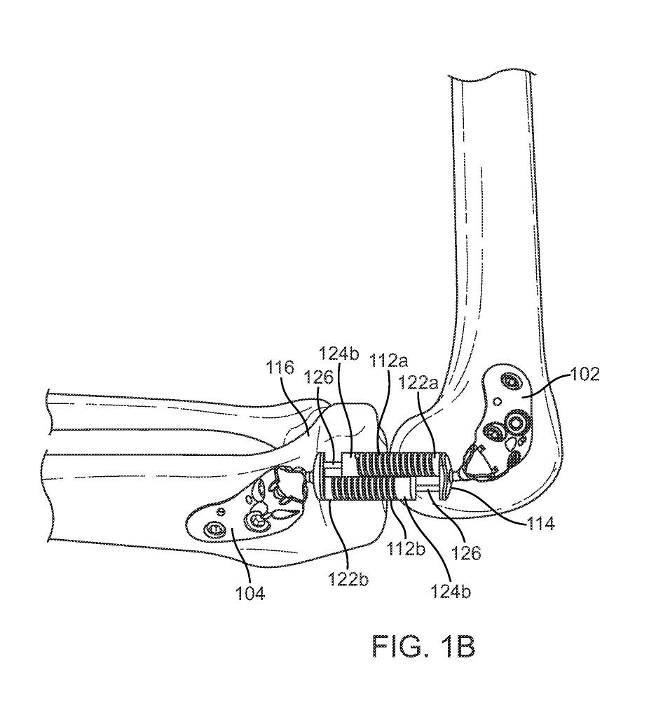

Referring now to FIGS. 1A and 1B, one embodiment of an energy absorbing system 100 is shown with proximal 102 and distal 104 bases secured upon first 106 and second 108 members, respectively of a typical body joint. Here, the terminal end portions of the femur and tibia are depicted without surrounding tissue. A more detailed description of the proximal and distal bases 102 and 104 can be found in U.S. application Ser. No. 12/755,335, filed Apr. 6, 2010, which is hereby incorporated by reference in its entirety. Also shown is an energy absorbing device 110 that is configured to provide unloading to the joint and is mounted between the bases. The energy absorbing device 110 includes a pair of springs 112a and 112b for biasing the proximal and distal bases 102, 104 and the associated joint members apart.

In one embodiment an implantable system for manipulating energy transferred by members defining a joint includes a first attachment structure or base 102 configured to be attached to a first member of the joint and a second attachment structure or base 104 configured to be attached to a second member of the joint. The energy absorbing device 110 is attachable to the first attachment structure and second attachment structure. The energy absorbing device 110 has a first part 114 with a piston and arbor and a second substantially identical part 116 having a piston and arbor. One or more springs 112a and 112b are located coaxial with the piston and arbor portions to provide unloading to the joint. The pistons and arbors are slidable with respect to one another to allow compression and extension of the energy absorbing device 110 in response to motion of the joint through a normal range of motion.

FIG. 1A shows the knee joint at full extension with load being applied to springs 112a and 112b of the energy absorbing device 110, whereas FIG. 1B shows the knee joint flexed to approximately 90° with zero load being applied to the springs by virtue of extension of the absorber 110. The energy absorbing device 110 lengthens as the knee swings from full extension (FIG. 1A) to flexion (FIG. 1B) and subsequently shortens as the knee swings back from flexion to full extension. At full extension, the springs 112a, 112b are compressed between the ends of the energy absorbing device 110 to absorb a portion of the load that the knee articulating surfaces normally would experience. As the joint moves into flexion, the springs 112a, 112b are gradually unloaded until at a predetermined flexion angle the springs are completely unloaded and free to float within the absorber 110.

The energy absorbing device 110 and bases 102, 104 are preferably mounted at the joint at a position and arrangement such that once the springs have achieved a predetermined amount of compression, and therefore load, the articulating surfaces of the knee then begin to carry the load in combination with the energy absorbing device such that the energy absorbing device does not “bottom out.” The energy absorbing devices in the present application are shown without a protective covering or sheath but it is contemplated that a protective covering or sheath can be placed over the absorber 110 and other portions of the system to protect the moving elements from impingement by surrounding tissues and to prevent the devices from damaging surrounding tissue. Examples of sheaths for energy absorbing devices are described in further detail in U.S. Patent Publication No. 20099/0275945 which is incorporated herein by reference in its entirety.

Still referring to FIGS. 1A and 1B, one embodiment of an energy absorbing device 110 includes a first absorber part 114 and a second absorber part 116. In one embodiment, the first and second absorber parts 114, 116 are generally identical to one another with one absorber part being rotated 180° from the other absorber part to mate and receive the springs 112a, 112b. The first absorber part 114 is connected to a first or proximal mount 118 that is in connection with the proximal or first base 102. On the other end of the energy absorbing device, the second absorber part 116 is connected to a second or distal mount 120 that is in connection with the distal or second base 104. Universal joints connect the absorber 110 to the proximal and distal mounts 118, 120. As shown, the first and second absorber parts 114, 116 each include ball ends 119 that fit within sockets of the proximal and distal mounts 118 and 120 to form the universal joints. The ball end of the first absorber part 114 is joined with a socket of the proximal mount 118 and the ball end of the second absorber part 116 is joined with a socket of the distal mount 120 to form ball and socket joints. The connection between the ball ends of the absorber and the mounts is disclosed in greater detail in U.S. application Ser. No. 12/755,335, which has already been incorporated by reference. Although the energy absorbing device 110 is shown to be connected to the first and second bases 102, 104 by ball and socket joints having three degrees of freedom, it should be understood that other connections may also be used and different connections may be used for connection to the two bases.

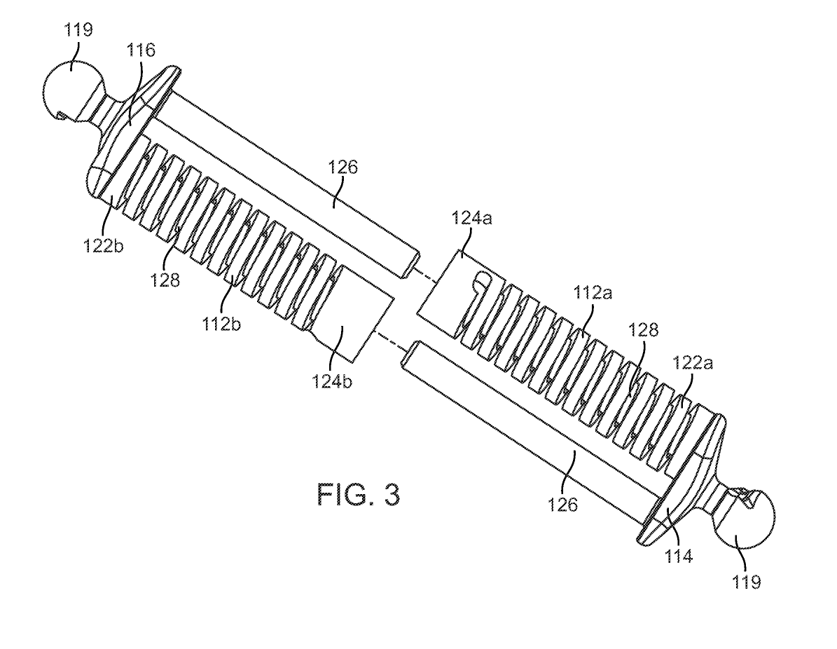

As shown most clearly in FIG. 3, first or proximal ends 122a and 122b of the springs 112a and 112b are in connection with or in contact with the first and second absorber parts, respectively. The proximal ends of the springs may be attached, welded, pinned, pressed, or the like, to the absorber ends. In another embodiment, the proximal ends 122a, 122b of the springs are not connected to the energy absorbing device 110 and are allowed to float freely on the arbors 128 between the ends of the first and second absorber parts 114, 116.

In one embodiment, the second or distal ends 123a and 123b of the springs 112a and 112b are in connection with or in contact with spacers 124a and 124b, respectively. The size of the spacers can be modified to affect the amount of compression of the springs. Also, the spacers can be formed of material providing compliance or spring behavior for added energy absorbing or as a built-in overload safety mechanism. The spacers 124a, 124b may be integral with or attached to the springs 112a, 112b. Alternatively, the spacers 124a, 124b may be eliminated to provide springs of greater lengths. According to one embodiment, the springs 112a, 112b can be provided in a selection of sizes to accommodate different profiles and can be either preassembled or assembled at the time of surgery taking into consideration patient weight, age, activity level, anatomy and other factors. When the springs are selected at time of surgery the surgeon can select the use of either one or two springs depending on the patient. The absorber will function with either one or two springs. In addition, two springs of different stiffness or lengths can be used to manipulate the absorption pattern as desired for a particular patient.

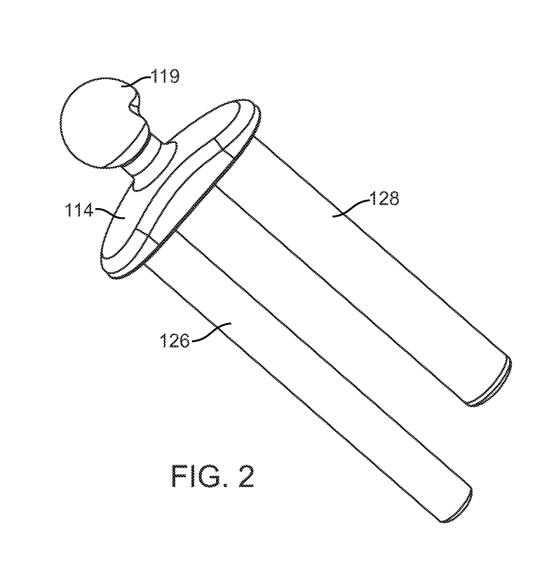

Referring now to FIG. 2, the first absorber part 114 includes a piston shaft 126 and an arbor 128 extending in a parallel arrangement from the end of the absorber part. The piston 126 and arbor 128 can be machined as part of the absorber or attached, pressed, welded, pinned, or otherwise attached to the absorber end. In the event that the absorber piston and arbor are formed of PEEK or other polymer material, these parts can be machined or molded. The piston 126 is sized and shaped to slide within the arbor 128 and the arbor is sized to receive the springs 112a, 112b coaxially arranged about the arbor. Although cylindrical shaped pistons 126, arbors 128 and springs 112a, 112b have been illustrated, other complementary shapes may also be used.

As shown in FIG. 3, the second absorber part 116 is identical or substantially identical to the first absorber part 114 and is rotated 180 degrees with respect to the first absorber part. To mate or engage the first and second absorber parts with one another, one of the absorber parts is rotated 180 degrees from the other absorber part and the pistons 126 are inserted into the arbors 128. In this embodiment, the piston shaft 126 of the second absorber part 116 will slide within the arbor 128 of the first absorber part, and the piston shaft 126 of the first absorber part 114 will slide within the arbor 128 of the second absorber part 116. During flexion and extension of the joint the pistons are slidable in the arbors to allow compression of the springs and to accommodate a changing distance between the bases 102, 104 of the system. In one embodiment, there is no mechanical limit to the extension of the first and second absorber parts. The absorber parts are prevented from completely disengaging with one another by the locations of implantation of the first and second bases 102, 104 and the anatomy of the joint.

In one contemplated embodiment, the energy absorbing device 100 can absorb at least a portion of a load found within a knee joint during at least five degrees and no more than sixty degrees of natural motion of the knee. For example, the device 100 can provide unloading from extension through at least five degrees and up to sixty degrees of flexion. Further, the first and second parts of the absorber can be configured to remain in a telescoping arrangement throughout a full range flexion of a joint.

Although two compression springs are shown in the energy absorbing device, only a single spring may be used. Alternatively, more than one spring may be placed on each of the pistons, such as when springs of different stiffnesses are used to provide a desired unloading pattern. The configuration of the springs may be varied to minimize device size while maximizing energy absorbing capabilities. Moreover, various types of springs can be used alone or in combination including coaxial springs, serial arranged springs, oval or other shaped springs, and springs having multiple parts with differing stiffnesses.

One embodiment of a spring can include a first stiffness that is active during normal operation of the energy absorbing device and a second region that has a stiffness greater than the stiffness of the first region. In this embodiment, the second region can be set such that it only becomes active when the first region of the spring reaches solid height. In that way, the second region can act to soften the stop if the energy absorbing device experiences loads (or displacements) higher than expected.

Biologically inert materials of various kinds can be employed in constructing the energy absorbing devices of the present invention. For example, the first and second energy absorbing parts and the springs can be titanium or titanium alloy, cobalt chromium alloy, ceramic, high strength plastic such as polyetheretherketone (PEEK) or other durable materials. Combinations of materials can also be used to maximize the properties of materials for different part so the device. At the wear surfaces, the material may include a combination of metal-on-poly, metal-on-metal, metal-on-ceramic or other combinations to minimize wear.

Certain members of the present invention can be made in multiple parts designed for modular assembly of different sizes and shapes and for easy removal and, if necessary replacement of some members or parts of members without removal of the entire system. For example, in the case of unanticipated wear, change in patient condition or availability of newer and improved parts, members can be removed and replace.

One example of a method for implantation of the energy absorbing device described herein involves an initial re-operative session to assess the need at a joint and to map the articulation of the members 106 and 108 forming the joint. Attachment sites can also be assessed pre-operatively. During surgical intervention spinal anesthesia or general anesthesia can be used. The knee or other joint being treated can be imaged using fluoroscopy and/or three-dimensional navigational software such as that available from Striker or Brainlab. Under visualization or based on pre-operative assessment, a first center of rotation location is identified along the first member of a joint. Next, access is gained to an area proximate the first center of rotation location and the first base 102 is fixed upon the first member in a manner maintaining use of the first center of rotation location. Subsequently, the second base 104 is fixed along the second member. A subcutaneous channel is created between the first and second base locations and the energy absorbing device 110 is inserted within the channel. The energy absorber is mounted to the bases with the center of rotation of the joint at the first end of the absorber located at a desired location with respect to the center or rotation of the joint which was previously determined. A tissue barrier, such as a sheath, may be placed about the energy absorber to protect joint anatomy or exclude the device from surrounding tissue. The connection of the absorber 110 to the bases 102 and 104 through optional attachable/detachable mounts 118 and 120 provides a simple surgical technique for installing the absorber. It also allows a sheath and/or the wear components of the absorber/mount assembly to be removable and/or replaceable without removing or replacing the bases.

In a contemplated method, the energy absorbing device 110 can be initially configured to eliminate or reduce loads to a desired degree, and to be later adjusted or altered as patient needs are better determined or change. Accordingly, post-operative alterations are contemplated as are adjustments resulting from changing the diameter of a dampening component or a spring rate of a device. It is further contemplated that the device may be considered temporary, being implanted for a defined service life and then removed or replaced. Additional details and other embodiments of an energy absorbing system and method of implantation are shown and described in U.S. Patent Publication Nos. 2008/0275571 and 2009/0014016, which were previously incorporated by reference.

The embodiments described herein for use in the knee can be for use throughout the body for unloading of articulating body structures such as joints. Although the present disclosure is particularly suited to treating synovial joints such as the knee, hip, ankle and shoulder, it is also contemplated that the apparatus and methods of the present disclosure can be employed to treat the spine facet joints and spine vertebral joints as well as other synovial and various other joints of the body such as those of the hand, wrist and feet.

The energy manipulation systems described herein can be implanted by conventional surgical or minimally invasive surgical approaches to the joints. Arthroscopic approaches are contemplated when reasonable to both implant the energy manipulation assembly as well as to accomplish adjusting an implanted assembly. Moreover, biologically inert materials of various kinds can be employed in constructing the energy manipulation assemblies of the present disclosure.

It will be apparent from the foregoing that, while particular forms of the embodiments have been illustrated and described, various modifications can be made without parting from the spirit and scope of the invention.

We claim:

1. An implantable system for manipulating energy transferred by members defining a joint, comprising:
a first attachment structure configured to be attached to a first member of the joint;
a second attachment structure configured to be attached to a second member of the joint; and
an energy absorbing device attachable to the first attachment structure and second attachment structure, wherein the energy absorbing device comprising a first part having a piston and arbor arranged parallel to one another and a second part having a piston and arbor of substantially the same configuration as the piston and arbor of the first part, wherein the piston of the first part is slidable within the arbor of the second part and wherein the piston of the second part is slidable within the arbor of the first part.

2. The system of claim 1, wherein the first part and the second part each include a portion of a universal joint and the first and second attachment structures each include a mating portion of the universal joint configured to mate with the first and second parts.

3. The system of claim 2, wherein the universal joint portion on the first and second parts comprise a ball of a ball and socket joint.

4. The system of claim 1, wherein the first part and the second part are substantially identical.

5. The system of claim 1, further comprising first and second springs configured to be received over the arbors of the first and second part.

6. The system of claim 1, wherein the implantable system is configured to be entirely implanted within a patient.

7. The system of claim 1, wherein the implantable system is configured to be implanted outside of the articular surfaces of the joint.

8. The system of claim 1, wherein the joint is a knee joint, the first and second attachment structures are configured to be attached to bones on opposite sides of the knee joint and the energy absorbing device is configured to span across the knee joint.

9. An implantable energy absorbing device comprising:
a first part having a piston and arbor arranged parallel to one another;
a second part having a piston and arbor of substantially the same configuration as the piston and arbor of the first part, wherein the piston of the first part is slidable within the arbor of the second part and wherein the piston of the second part is slidable within the arbor of the first part; and
at least one spring received over an arbor of the first or second part and configured to bias the first and second parts apart.

10. The energy absorbing device of claim 9, wherein the energy absorbing device is configured to span across a joint.

11. The energy absorbing device of claim 10, wherein the joint is a knee joint.

12. The energy absorbing device of claim 9, wherein the first and second parts are substantially identical.

13. The energy absorbing device of claim 12, wherein the first and second parts are each unitary parts and are translatable with respect to one another.

14. The energy absorbing device of claim 12, wherein the at least one spring comprises two springs received over the arbors of the first and second parts.

15. An implantable energy absorbing device comprising:
a first part having a piston and arbor arranged parallel to one another;
a second part having a piston and arbor of substantially the same configuration as the piston and arbor of the first part, wherein the piston of the first part is slidable within the arbor of the second part and wherein the piston of the second part is slidable within the arbor of the first part; and
wherein the first and second parts are substantially identical and rotated 180 degrees with each other to mate in a sliding arrangement.

16. The energy absorbing device of claim 15, wherein the arbors of the first and second parts are formed at least in part as springs.

17. The energy absorbing device of claim 15, further comprising two springs received over the arbors of the first and second part and configured to bias the first and second parts apart.

18. A method of assembling an implantable energy absorbing device, the method comprising:
providing a first part having a piston and arbor arranged parallel to one another;
providing a second part having a piston and arbor of substantially the same configuration as the piston and arbor of the first part;
positioning two springs over the arbors of the first and second part;
inserting the piston of the first part within the arbor of the second part and the piston of the second part within the arbor of the first part; and
biasing the first and second parts apart with the two springs.

19. An implantable system for manipulating energy transferred by members defining a joint, comprising:
a first attachment structure configured to be attached to a first member of the joint;
a second attachment structure configured to be attached to a second member of the joint; and
an energy absorbing device attachable to the first attachment structure and second attachment structure, wherein the energy absorbing device comprising a first part having a first member and a second member arranged parallel to one another and a second part having a first member and a second member of substantially the same configuration as the first and second members of the first part, wherein the first member of the first part is slidable relative to the second member of the second part.

20. The system of claim 19, further comprising first and second springs configured to be received over the second members of the first and second part.

21. The system of claim 19, wherein the implantable system is configured to be entirely implanted within a patient.

22. The system of claim 19, wherein the implantable system is configured to be implanted outside of the articular surfaces of the joint.

23. The system of claim 19, wherein the joint is a knee joint, the first and second attachment structures are configured to be attached to bones on opposite sides of the knee joint and the energy absorbing device is configured to span across the knee joint.

24. The system of claim 19, wherein the first member of the first part is slidable inside the second member of the second part and wherein the first member of the second part is slidable inside the second member of the first part.
